(19) United States
(12) Patent Application Publication
(10) Pub. No.: US 2002/0099331 A1
(43) Pub. Date: Jul. 25, 2002

(54) Sinus and nasal irrigating apparatus

(76) Inventor: Timothy J. Burchfield, Glens Falls, NY (US)

Correspondence Address: BILL B. BERRYHILL, Suite 280, 6300 West Loop South, Bellaire, TX, 77401, US

(21) Appl. No.: 09/768,545

(22) Filed: Jan. 25, 2001

(51) Int. Cl. A61M 31/00

(52) U.S. Cl. 604/94.01

(57) ABSTRACT

Apparatus for irrigating a person's sinuses and nasal passages which includes a container having one or more openings through which the container may be filled with water or other fluids; a flexible tubular member one end of which is attached to a container opening; a nozzle attachable to the other end of the tubular member for insertion into a selected nostril of the person; and support for the container in an elevated position.

### BACKGROUND OF THE INVENTION

1. Field of the Invention

The present invention pertains to sinus and nasal irrigating apparatus. More specifically, the present invention pertains to apparatus for irrigating the sinuses and nasal passages by irrigating with water or other fluids through a person's nostrils.

2. Description of the Prior Art

It is well known that irrigation of the nostrils with warm water or other fluids is beneficial in clearing nose passages and providing relief to inflamed sinuses and/or other nasal tissues. Probably the simplest apparatus utilized for such is a syringe in which the nozzle thereof is inserted into the nose for irrigation with water or other fluids contained in the syringe.

In recent years, a number of devices have been developed which are more effective than the simple syringe. For example, U.S. Pat. No. 3,847,145 discloses a nasal irrigation system which has a hose and nozzle or applicator through which a water solution is introduced into a nostril. The hose is connected to a pulsating pressure device through which water is pulsed. The nozzle or applicator is provided with several components for controlling flow of the pulsating water. U.S. Pat. No. 5,899,878 discloses a similar nasal irrigation system in which flow of fluids is controlled by covering and uncovering an opening in the nozzle or applicator. Both of these systems are relatively complex and require a source of pressurized, and preferably pulsating, water or fluid.

### SUMMARY OF THE PRESENT INVENTION

The present invention provides apparatus for irrigating nasal passages to relieve inflamed sinuses and other nasal tissues. It includes a container which may be filled with water or other fluids, a flexible tubular member one end which may be attached to the container and a nozzle which may be attached to the other end of the tubular member. The nozzle is adapted for inserting into a selected nostril of the person using the apparatus. The apparatus also includes means for supporting the container at an elevation at least as great as the person's sinuses, allowing the water or other fluids in the container to flow by gravity into the person's nostril.

The irrigating apparatus of the present invention does not require a pulsating pressure source, as with those of the prior art discussed above. Consequently, it is less complicated and may be provided at a much reduced cost. However, it is easy to use and efficient in irrigating the sinuses and nasal passages. Other objects and advantages of the present invention will be apparent from reading the description which follows in conjunction with the accompanying drawings.

### BRIEF DESCRIPTION OF THE DRAWINGS

FIG. 1 is a pictorial illustration of a person utilizing the irrigating apparatus of the present invention; and

FIG. 2 is an elevation view of the irrigating apparatus of the present invention, showing the components thereof in more detail.

### DESCRIPTION OF A PREFERRED EMBODIMENT

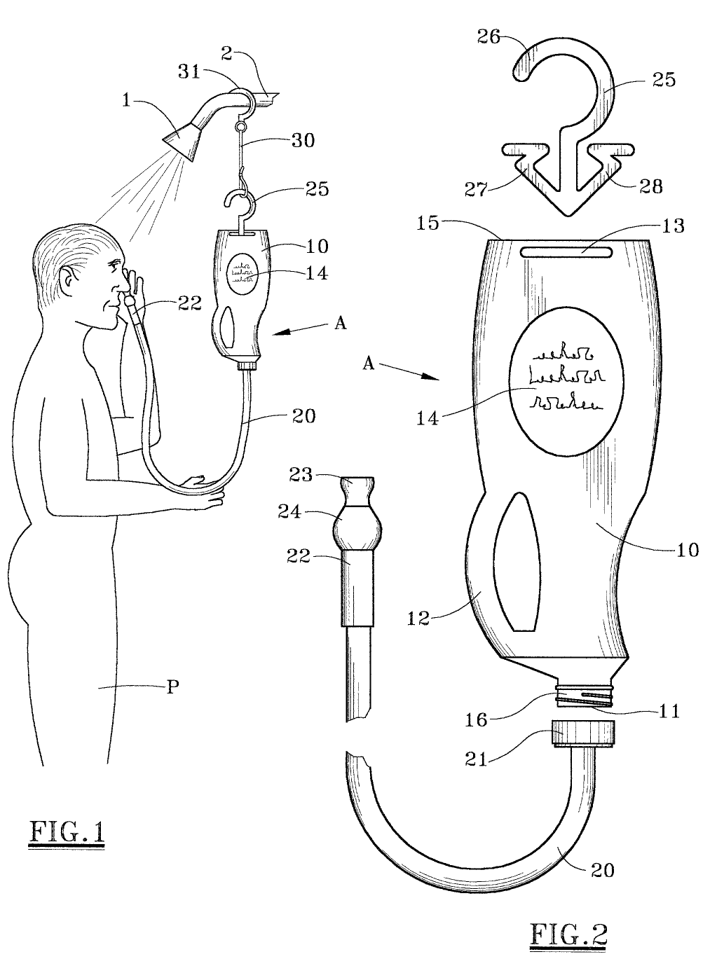

Referring first to FIG. 1, there is shown a person P, illustrated as being in a shower which includes a shower nozzle 1 attached to piping 2 which would normally be connected to a source of water. Hanging from the piping 2 is irrigating apparatus A which will be described in greater detail hereafter.

As shown in FIG. 2, the irrigating apparatus A comprises a container 10 having an upper opening 15 therein through which the container 10 may be filled with warm water or other fluids. When initially filled and used, the container 10 would be in the positions illustrated in FIG. 1 and FIG. 2. The container 10 may be provided with a handle 12 of any kind for aiding in lifting the container 10. The container 10 also has a lower opening 11. A flexible tubular member 20 approximately three feet long, and preferably of a transparent material, is provided with a threaded fitting 21 which cooperates with a corresponding fitting, threads 16 in the exemplary embodiment, surrounding the container opening 11, allowing the tubular member 20 to be sealingly and removably attached to the container opening 11.

Provided at the opposite end of the tubular member 20 is a nozzle 22 which may be flared at 23 and is adapted for insertion into a selected nostril of a person. A soft bulbous rubber sleeve attachment 24 may be fitted around the nozzle to aid in sealing with the nostril. The nozzle 22 is removably attached to the tube 20 so that it may be removed for cleaning and storage after use.

A hook member 25 may be provided for supporting the container from a fixed support, such as the pipe 2 in FIG. 2. The hook member 25 may be simply a curved hook one terminal end 26 of which maintains the hook 25 in position from a support by which the apparatus A is suspended. The opposite end of the hook member 25 may be provided with one or more, two in the exemplary embodiment, arms or extensions 27, 28 which are engageable with the slot 13 in the container 10 to support the container 10 at an elevated position, such as shown in FIG. 1. The arms 27, 28 on each side of the hook member 25 allows engagement of the slot 13 for any container orientation.

Referring now to both figures, use of the irrigation apparatus A of the present invention will be described. The apparatus is best suited for use in a shower or tub but may also be used over a sink. It is self-administered and should be used only by a responsible adult.

To begin, the container 10, with the hook 25 in place, is filled with lukewarm tap water or, if preferred, lukewarm distilled water. A small amount of sea salt, e.g. one-half teaspoon per quart of water, may be added. The tube 20, with nozzle 22 has already been attached to the container 10 with the cooperating fittings 16, 21. The container 10 is then suspended by the hook 25 from an adjustable strap 30 and corresponding hook 31 so that the label 14 is essentially at eye level, as shown in FIG. 1. Alternatively, the hook 25 may be suspended from a self-adhering wall hook (not shown) which could be adhered to an adjacent wall. Instructions for use could be provided on the label 14. As stated, the label 14 of the container 10 would be placed at sinus level to insure proper water pressure.

Some water should be allowed to flow out of the tube 20 to insure that there are no air bubbles in the tube which might impede flow of fluid out of the nozzle 22. Since the tube 20 is transparent it may be easily inspected for air bubbles. It is very important that there not be any air in the tube since air could cause injury if forced into the sinuses under pressure.

The tip 23 of the nozzle 22 would then be inserted in one nostril of the user P. Using a free hand, the user P would press the outside of the nostril to form a seal around the nozzle sleeve 24. The water or other fluid in the bottle would then flow, by gravity, into the nasal and sinus cavities and drain out of the other nostril. The mouth should be opened during this operation to equalize pressure to the ears. The mouth should continue to be open, the user breathing through his mouth and being careful not to place pressure on the ears. If pressure is felt on the eardrums, the nozzle should be removed immediately to stop the procedure. Pressure in the ears should be equalized before resuming the procedure.

If it is desired to increase pressure while performing the procedure, the user may bend forward. Conversely, if it is desired to reduce the pressure, the user would stand upright. As the container 10 empties, water pressure from the nozzle 22 will, of wall hook (not shown) which could be adhered to an adjacent wall. Instructions for use could be provided on the label 14. As stated, the label 14 of the container 10 would be placed at sinus level to insure proper water pressure.

Some water should be allowed to flow out of the tube 20 to insure that there are no air bubbles in the tube which might impede flow of fluid out of the nozzle 22. Since the tube 20 is transparent it may be easily inspected for air bubbles. It is very important that there not be any air in the tube since air could cause injury if forced into the sinuses under pressure.

The tip 23 of the nozzle 22 would then be inserted in one nostril of the user P. Using a free hand, the user P would press the outside of the nostril to form a seal around the nozzle sleeve 24. The water or other fluid in the bottle would then flow, by gravity, into the nasal and sinus cavities and drain out of the other nostril. The mouth should be opened during this operation to equalize pressure to the ears. The mouth should continue to be open, the user breathing through his mouth and being careful not to place pressure on the ears. If pressure is felt on the eardrums, the nozzle should be removed immediately to stop the procedure. Pressure in the ears should be equalized before resuming the procedure.

If it is desired to increase pressure while performing the procedure, the user may bend forward. Conversely, if it is desired to reduce the pressure, the user would stand upright. As the container 10 empties, water pressure from the nozzle 22 will, of course, decrease. After emptying, the container 10 may be refilled and the procedure repeated, placing the nozzle 22 in the other nostril, if desired. The procedure may be continued until the user is satisfied that the nasal passages and sinuses are clear or sufficiently moisturized. The user may wish to repeat the procedures after an hour or two, depending on severity of sinus complications and other circumstances.

The apparatus A should be thoroughly cleaned after each use. It may be cleaned with a mild bleach solution and allowed to dry by hanging. The nozzle should especially be well cleaned and dried. It can be stored in a sterile or clean container.

The irrigating apparatus of the present invention is very efficient and simple to operate, pressure therefor being provided by gravity. It is much less complicated than devices of the prior art and can therefore be provided at a much reduced cost.

A single embodiment of the invention, and the use thereof, has been described herein. Many variations thereof can be made by those skilled in the art, without departing from the spirit of the invention. Accordingly, it is intended that the scope of the invention be limited only by the claims which follow.

1. Apparatus for irrigating a person's sinuses comprising:
a container having one or more openings therein and through one of which said container may be filled with water or other fluids;
a flexible tubular member one end of which may be attached to one of said openings;
a nozzle which may be attached to another end of said tubular member and adapted for inserting into a selected nostril of said person; and
means for supporting said container at an elevation at least as great as said person's sinuses to allow said water or other fluids to flow by gravity into said person's nostril.

2. Sinus irrigating apparatus as set forth in claim 1 in which said one of said container openings and said one end of said flexible tubing are provided with cooperating fittings by which said container and said tubular member may be sealingly and removably attached.

3. Sinus irrigating apparatus as set forth in claim 1 in which said nozzle is provided with a soft rubber-like bulbous sleeve for sealing engagement with said selected nostril.

4. Sinus irrigating apparatus as set forth in claim 1 in which said nozzle is removable from said tubular member for cleaning and storage after use.

5. Sinus irrigating apparatus as set forth in claim 1 in which said means for supporting said container comprises a hook member by which said apparatus may be suspended near the place of use by said person.

6. Sinus irrigation apparatus as set forth in claim 5 in which said hook member is removably attachable to said container.
